(19) United States
(12) Patent Application Publication
(10) Pub. No.: US 2004/0115213 A1
(43) Pub. Date: Jun. 17, 2004

(54) Use of IL-2 receptor antibodies

(76) Inventors: Stanley Willetts Chapman, Jackson, MS (US); John Dale Cleary, Brandon, MS (US); Robert D. Gordon, Atlanta, GA (US)

Correspondence Address: HOFFMANN-LA ROCHE INC., PATENT LAW DEPARTMENT, 340 KINGSLAND STREET, NUTLEY, NJ, 07110

(21) Appl. No.: 10/677,148

(22) Filed: Oct. 1, 2003

Related U.S. Application Data
(60) Provisional application No. 60/416,302, filed on Oct. 4, 2002.

(51) Int. Cl. A61K 39/00; A61K 39/38

(52) U.S. Cl. 424/184.1

(57) ABSTRACT

The invention relates to a method for treating a patient having a fungal infection. The invention comprises administering to the patient a therapeutically effective amount of a polyene antifungal (e.g., amphotericin B) in association with a therapeutically effective amount of a interleukin-2 inhibitor (e.g., chimeric or humanized monoclonal antibody that binds to the p55 subunit of the human interleukin-2 receptor and inhibits binding of interleukin-2 to an interleukin-2 receptor).

### FIELD OF THE INVENTION

The invention relates to the use of an interleukin-2 (“IL-2”) inhibitor, e.g., (a monoclonal antibody that binds to the p55 subunit of the human interleukin-2 (“IL-2”) receptor of human T lymphocytes), to prevent toxicity associated with the use of polyene antimycotics (e.g., amphotericin-B, lipid formulations of amphotericin B or nystatin) in the treatment of systemic fungal infections.

### BACKGROUND OF THE INVENTION

Amphotericin-B is a polyene antimycotic used to treat invasive fungal infections. It is highly effective and is frequently used in critically ill patients with life threatening illnesses. Patients who receive amphotericin-B commonly experience several reactions including: renal dysfunction with secondary hypokalemia, hypomagnesemia and anemia, flu-like syndrome (headaches, fever, chills, malaise, myalgias, joint pain, anorexia, weight loss, nausea and vomiting), and epigastric pain. Other side effects include cardiovascular toxicity (hypotension, ventricular fibrillation, or cardiac arrest), pulmonary leukocytosis (dyspnea, respiratory distress) hepatic dysfunction or failure, coagulation defects, pruritis, and seizures. Over 80% of patients experience some degree of nephrotoxicity when given amphotericin-B.

### SUMMARY OF THE INVENTION

The invention provides methods of using a monoclonal antibody that binds to the p55 subunit of the human interleukin-2 (“IL-2”) receptor of human T lymphocytes, to prevent toxicity associated with the use of an antifungal agent, such as amphotericin-B formulations, in the treatment of systemic fungal infections.

Such methods include treating a patient having a fungal infection, and comprise the administration to said patient of a therapeutically effective amount of an antifungal agent with a therapeutically effective amount of a chimeric or humanized monoclonal antibody that binds to the p55 subunit of the human interleukin-2 (IL-2) receptor and inhibits binding of IL-2 to an IL-2 receptor. In particular, such methods entail treating a patient with a therapeutically effective amount of amphotericin B formulation in association with a therapeutically effective amount of a chimeric or humanized monoclonal antibody that binds to the p55 subunit of the human interleukin-2 (IL-2) receptor and inhibits binding of IL-2 to an IL-2 receptor.

In another aspect, such methods entail treating a patient having a fungal infection, and comprise the administration over a given period of time to the patient of (i) a first component consisting of a pharmaceutical composition consisting of an injectable solution containing as an active ingredient an antimycotic, such as an amphotericin B formulation, in a therapeutically effective amount to decrease the severity of the fungal infection and (ii) a second component consisting of an injectable solution containing as an active ingredient a chimeric or humanized monoclonal antibody that binds to the p55 subunit of the human interleukin-2 (IL-2) receptor and inhibits binding of IL-2 to an IL-2 receptor in a therapeutically effective amount to prevent reactions commonly experienced by patients who receive an amphotericin B formulation or other such antifungal agent, said components being concomitantly administered over a period of time at least sufficient to permit continued therapy with an amphotericin formulation or other such antifungal agent without intolerable or life threatening side effects. Alternatively, the first component can be any pharmacological agent that inhibits IL-2 or IL-2 receptor actions and the second component can be any polyene antimycotic.

In yet another aspect, methods of the invention entail treating a patient having a fungal infection, and comprise concomitantly administering to the patient (i) a first component comprising an injectable solution containing, as an active ingredient, an antimycotic and (ii) a second component comprising an injectable solution containing as an active ingredient a chimeric or humanized monoclonal antibody that binds to the p55 subunit of the human interleukin-2 (IL-2) receptor and inhibits binding of IL-2 to an IL-2 receptor. In particular, these methods comprising concomitantly administering to the patient (i) a first component consisting of an injectable solution containing as an active ingredient amphotericin B, and (ii) a second component consisting of an injectable solution containing as an active ingredient a chimeric or humanized monoclonal antibody that binds to the p55 subunit of the human interleukin-2 (IL-2) receptor and inhibits binding of IL-2 to an IL-2 receptor. More particularly, such methods entail treating a patient having a fungal infection, comprising concomitantly administering to the patient (i) a first component consisting of an injectable solution containing as an active ingredient amphotericin B, wherein the active ingredient of the first component is administered in an approximate amount of 0.3 mg/kg to 25 mg/kg per day, and (ii) a second component consisting of an injectable solution containing as an active ingredient a chimeric or humanized monoclonal antibody that binds to the p55 subunit of the human interleukin-2 (IL-2) receptor and inhibits binding of IL-2 to an IL-2 receptor wherein the active ingredient of the second component is administered in an approximate amount of 1 mg/kg to 2 mg/kg per week, said components being concomitantly administered over a period of time from about 4 weeks to about 8 weeks.

In yet another aspect, methods of the invention entail treating a patient having a fungal infection, and comprise administering to the patient over a period of time at least sufficient to resolve an invasive fungal infection, in at least fourteen day cycles, (a) a first component, delivered on day 1 of each fourteen day cycle, consisting of an injectable solution containing as an active ingredient a chimeric or humanized monoclonal antibody that binds to the p55 subunit of the human interleukin-2 (IL-2) receptor and inhibits binding of IL-2 to an IL-2 receptor in a therapeutically effective amount to prevent reactions commonly experienced by patients who receive an antifungal agent, and (b) a second component, delivered via multiple regimens, consisting of an injectable solution containing as an active ingredient an antifungal agent in a therapeutically effective amount to decrease the severity of the fungal infection. Preferably the monoclonal antibody is an anti-Tac antibody, such as daclizumab and the antifungal agent is an antimycotic, such as an amphotericin B formulation.

In another aspect, methods of the invention entail reducing side effects associated with antifungal therapy selected from the group consisting of renal dysfunction with secondary hypokalemia, hypomagnesemia and anemia, flu-like syndrome, epigastric pain, cardiovascular toxicity, hypotension, ventricular fibrillation, cardiac arrest, pulmonary leukocytosis, dyspnea, respiratory distress, hepatic dysfunction or failure, coagulation defects, pruritis, seizures, and nephrotoxicity comprising administering to a patient a therapeutically effective amount of a chimeric or humanized monoclonal antibody that binds to the p55 subunit of the human interleukin-2 (IL-2) receptor and inhibits binding of IL-2 to an IL-2 receptor.

In yet another aspect, the instant invention entails a kit comprising (i) a first component containing a vial or series of vials, each vial containing a single injectable solution dose or multiple injectable solution doses, each dose containing an active ingredient about 50 mg to 100 mg of the active ingredient, wherein the active ingredient is amphotericin B, and (ii) a second component containing a vial or series of vials, each vial containing a single injectable solution dose or multiple injectable solution doses, each dose containing as an active ingredient about 25 mg of a chimeric or humanized monoclonal antibody that binds to the p55 subunit of the human interleukin-2 (IL-2) receptor and inhibits binding of IL-2 to an IL-2 receptor.

### DETAILED DESCRIPTION OF THE INVENTION

The invention provides methods of using a pharmacological agent that interferes with IL-2 activity, such as a monoclonal antibody that binds to the p55 subunit of the human IL-2 receptor of human T lymphocytes, to prevent toxicity associated with the use of polyene antifungals, such as amphotericin-B formulations, in the treatment of systemic fungal infections. Preferably used as the pharmacological agent that interferes with IL-2 activity is a monoclonal antibody, more particularly a humanized or chimeric antibody or other antibodies produced by genetic engineering.

In particular, a patient having a fungal infection is administered a therapeutically effective amount of an antifungal agent, such as an amphotericin B formulation, in association with a therapeutically effective amount of a chimeric or humanized monoclonal antibody that binds to the p55 subunit of the human interleukin-2 (IL-2) receptor and inhibits binding of IL-2 to an IL-2 receptor. Preferably, the infection is a mycotic infection. Preferably, the monoclonal antibody is a humanized anti-Tac antibody, such as daclizumab.

“Therapeutically effective amount” means an amount that is effective to prevent, alleviate or ameliorate symptoms of disease or prolong the survival of the subject being treated.

The invention will further be described in terms of amphotericin B formulations and daclizumab. One having ordinary skill in the art will recognize that the invention can be practiced with any antifungal agent and any monoclonal antibody that binds to the p55 subunit of the human interleukin-2 (IL-2) receptor and inhibits binding of IL-2 to an IL-2 receptor.

In a preferred embodiment, administration of the therapeutically effective amount of amphotericin B and the therapeutically effective amount of daclizumab occurs concomitantly.

As used herein, the term “concomitantly” refers to administration within the same course of treatment. For example, concomitant administration includes administration of each component at the same time. Concomitant administration also includes administration of each component on a different schedule over the same period of time; e.g. administration of the monoclonal antibody once a week over the course of treatment and administration of the antifungal agent daily over the course of treatment. Concomitant administration also includes administration of the monoclonal antibody component prior to, during, or after administration of a course of antifungal agent.

Alternatively, a part of the therapeutically effective amount of daclizumab may be first administered followed by a combination of the remainder of the therapeutically effective amount of daclizumab in association with the therapeutically effective amount of amphotericin B. Alternatively, all of the therapeutically effective amount of daclizumab may be first administered followed by a therapeutically effective amount of amphotericin B.

Preferably, the amphotericin B is administered intravenously, for example, in an approximate amount of 0.3 mg to 25 mg/kg per day. More preferably, the amphotericin B is administered in an approximate amount of 0.3 mg to 25 mg/kg/day. Preferably, daclizumab is administered intravenously, for example in an amount of about 1 mg/kg four times per week.

More particularly, a patient having a fungal infection is administered over a given period of time (a) a first component consisting of pharmaceutical composition consisting of an injectable solution containing as an active ingredient amphotericin B in a therapeutically effective amount to decrease the severity of the fungal infection and (b) a second component consisting of an injectable solution containing as an active ingredient a chimeric or humanized monoclonal antibody that binds to the p55 subunit of the human interleukin-2 (IL-2) receptor and inhibits binding of IL-2 to an IL-2 receptor in a therapeutically effective amount to prevent reactions commonly experienced by patients who receive amphotericin B. The components are concomitantly administered over a period of time at least sufficient to permit maintaining administration of a clinically effective dose of amphotericin B for the time needed to control invasive fungal infection.

In a preferred embodiment, the second component consists of an injectable solution containing as an active ingredient a humanized anti-Tac monoclonal antibody, such as daclizumab.

A patient having a fungal infection may be treated by concomitantly administering to the patient: (i) a first component consisting of an injectable solution containing as an active ingredient amphotericin B, wherein the active ingredient of the first component is administered in an approximate amount of 0.3 mg/kg to 25 mg/kg per day, and (ii) a second component consisting of an injectable solution containing as an active ingredient a chimeric or humanized monoclonal antibody that binds to the p55 subunit of the human interleukin-2 (IL-2) receptor and inhibits binding of IL-2 to an IL-2 receptor wherein the active ingredient of the second component is administered in approximate amount of 1 mg/kg to 2 mg/kg per week. The components are concomitantly administered over a period of time from about 4 weeks to about 8 weeks.

Alternatively, the invention comprises treating a patient, having a fungal infection, by administering to the patient over a period of time at least sufficient to resolve an invasive fungal infection, in fourteen day cycles, (a) a first component, delivered on day 1 of each fourteen day cycle, consisting of an injectable solution containing as an active ingredient daclizumab in a therapeutically effective amount to prevent reactions commonly experienced by patients who receive an antifungal agent, and (b) a second component, delivered each day of each fourteen day cycle, consisting of pharmaceutical composition consisting of an injectable solution containing as an active ingredient amphotericin B in a therapeutically effective amount to decrease the severity of the fungal infection.

The invention also encompasses a method of reducing side effects associated with antifungal therapy. For example, the invention includes reducing side effects common to treatment with an amphotericin B formulation, such as renal dysfunction with secondary hypokalemia, hypomagnesemia and anemia, flu-like syndrome, epigastric pain, cardiovascular toxicity, hypotension, ventricular fibrillation, cardiac arrest, pulmonary leukocytosis, dyspnea, respiratory distress, hepatic dysfunction or failure, coagulation defects, pruritis, seizures, and nephrotoxicity. Such methods comprise administering to a patient a therapeutically effective amount of daclizumab.

In another aspect, the invention relates to a kit comprising (a) a first component containing a vial or series of vials, each vial containing a single injectable solution dose or multiple injectable solution doses, each dose containing an active ingredient about 0.3 to about 25 mg/kg of the active ingredient, wherein the active ingredient is amphotericin B, and (b) a second component containing a vial or series of vials, each vial containing a single injectable solution dose or multiple injectable solution doses, each dose containing as an active ingredient about 1 to about 2 mg/kg of a chimeric or humanized monoclonal antibody that binds to the p55 subunit of the human IL-2 receptor and inhibits binding of IL-12 to an IL-12 receptor.

Preferably, the first component contains a sufficient number of units so that a patient can administer about 0.3 mg/kg to about 25 mg/kg per day, more preferably about 0.3 to mg/kg to about 10 mg/kg per day, of amphotericin B for a period of about 4 to about 8 weeks and the second component contains a sufficient number of doses so that a patient can administer about 1 mg per kg per week of the monoclonal antibody for a period of about 4 to about 8 weeks.

More preferably, the active ingredient of each injectable solution dose of the second component is a humanized anti-Tac antibody, such as daclizumab.

In a preferred embodiment, the first component contains a sufficient number of units so that a patient can administer about 0.3 mg/kg to about 25 mg/kg per day of amphotericin B for a period of about 4 to about 8 weeks and the second component contains a sufficient number of doses so that a patient can administer about 1 mg per kg per week of the monoclonal antibody for a period of about 4 to about 8 weeks.

In another preferred embodiment, the first component contains a sufficient number of units so that a patient can administer about 0.3 mg/kg to about 10 mg/kg per day of amphotericin B for a period of about 4 to about 8 weeks and the second component contains a sufficient number of doses so that a patient can administer about 1 mg per kg per week of the monoclonal antibody for a period of about 4 to about 8 weeks.

Amphotericin B

Amphotericin B is a prototype macrocyclic, polyene, antifungal antibiotic produced from a strain of Streptomyces nodosus. For example, amphotericin B is produced by Streptomyces nodosus which has been deposited as ATCC#14899 by E. R. Squibb & Sons, Inc. It acts by binding to sterols (primarily ergosterol) in cell membranes of sensitive fungi, with subsequent leakage of intracellular contents and cell death due to changes in membrane permeability. Amphotericin B also binds to the sterols (primarily cholesterol) in mammalian cell membranes, which is believed to account for its toxicity in animals and humans.

Amphotericin B, which is the established name for [1R-(1 R *,3 S *, 5 R *, 6 R *,9 R *,11 R *,15 S *,16 R *,17 R *,18 S *, 19 E,21 E, 23 E,25 E, 27 E,29 E,31 E,33 R *,35 S *,36 R *,37 S *)]-33-[(3-Amino-3,6-dideoxy-(beta)-D-mannopyranosyl)oxy]-1,3,5,6,9,11,17,37-octahydroxy-15,16,18-trimethyl-13-oxo-14,39-dioxabi-cyclo[33.3.1]nonatriaconta-19,21,23,25,27,29,31-heptaene-36-carboxylic acid, has the following structure:

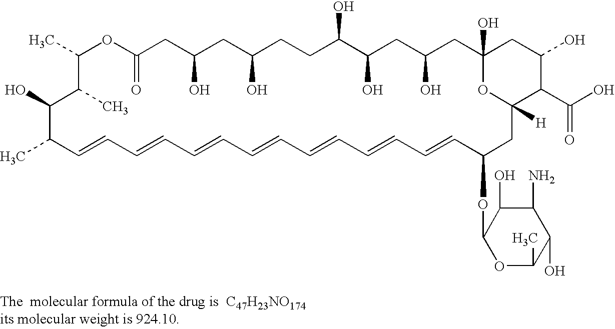

IL-2 Inhibitors—Antibodies

Specificity and Affinity

Monoclonal antibodies useful in the claimed methods typically bind to the p55 subunit of the IL-2 receptor with an affinity of at least 10⁸ M⁻¹ and preferably 10⁹ M⁻¹ or greater. Such monoclonal antibodies are typically humanized or chimeric antibodies, or are otherwise produced by genetic engineering methods. Preferred monoclonal antibodies bind to the same or overlapping epitope as the anti-Tac and humanized anti-Tac antibodies. Two antibodies bind to the same or overlapping epitope if each competitively inhibits (blocks) binding of the other to the antigen. That is, 1×, 5×, 10×, 20×x or 100× excess of one antibody inhibits binding of the other by at least 50% but preferably 75%, 90% or even 99% as measured in a competitive binding assay (see e.g., Junghans et al., Cancer Res. 1990:50:1495-1502). Alternatively, two antibodies have the same epitope if Essentially all amino acid mutations in the antigen that reduce or eliminate binding of one antibody reduce or eliminate binding of the other. Two antibodies have overlapping epitopes if some amino acid mutations that reduce or eliminate binding of the antibody reduce or eliminate binding of the other.

Monoclonal antibodies useful in the invention block binding of IL-2 to the IL-2 receptor or its p55 subunit. That is, addition of the antibody at a concentration of 0.1, 0.5, 1, 2, 5, 10 or 20 μg/ml inhibits binding of IL-2 to the p55 subunit or IL-2 receptor on suitable cells (e.g., HuT-102, YT-S2, or PHA blasts) by about at least 50% but preferably 75%, 90% or even 99%, as assayed by methods well known in the art (see Hakimi et al., J. Immunol. 1993:151:1075-1085 and Junghans et al., supra, both of which are herein incorporated by reference). Preferred monoclonal antibodies at concentrations of 1, 5, 10 or 20 μg/ml inhibit or block IL-2-dependent proliferation of appropriate cells by 50%, 75%, 90% or greater, for example of PHA blasts, or PBMC stimulated by tetanus toxoid or other antigen or mixed lymphocyte reaction (MLR), as assayed by art-known techniques (Hakimi et al., Junghans et al., supra).

Examples of antibodies, binding to the p55 subunit of the human interleukin-2 (IL-2) receptor of human T lymphocytes, and useful in the invention include chimeric anti-Tac antibody, described in patent application PCT/US89/01578; RFT5 chimeric antibody, described in EP 449 769 B1; BT563 described in Nasham, et al., Transplantation, 1996: 61: 546-554; a chimeric or humanized form of antibody 33B3.1 (Soulillou et al., New Eng. J. Med. 1990:322:1175-1182); and most preferably, humanized anti-Tac described in U.S. Pat. No. 5,530,101, incorporated herein by reference, or other humanized versions of anti-Tac. Other such antibodies can be produced by standard immunological and genetic engineering techniques.

General Characteristics

Antibodies are very large, complex molecules (molecular weight of 150,000 or about 1320 amino acids) with intricate internal structure. A natural antibody molecule contains two identical pairs of polypeptide chains, each pair having one light chain and one heavy chain. Each light chain and heavy chain in turn consists of two regions: a variable (“V”) region involved in binding the target antigen, and a constant (“C”) region that interacts with other components of the immune system. The light and heavy chain variable regions fold up together in 3-dimensional space to form a variable region that binds the antigen (for example, a receptor on the surface of a cell). Within each light or heavy chain variable region, there are three short segments (averaging 10 amino acids in length) called the complementary determining regions (“CDRs”). The six CDRs in an antibody variable domain (three from the light chain and three from the heavy chain) fold up together in 3-D space to form the actual antibody binding site which locks onto the target antigen. The position and length of the CDRs have been precisely defined. Kabat, E. et al., U.S. Department of Health and Human Services (1983): Chothia et al., J. Mol. Biol., 196:901 (1987) (the definitions of CDRs provided by Kabat and by Chothia are somewhat different). The part of a variable region not contained in the CDRs is called the framework, which forms the environment for the CDRs.

A humanized antibody is a genetically engineered antibody in which the CDRs (hereinafter reference to CDR can include both the Kabat and Chothia CDRs) from a mouse antibody (“donor antibody”, which can also be rat, hamster or other similar species) are grafted onto a human antibody (“acceptor antibody”). Thus, a humanized antibody is an antibody having CDRs from a donor antibody and variable region framework and constant regions from a human antibody. In addition, in order to retain high binding affinity, at least one of two additional structural elements can be employed. See U.S. Pat. Nos. 5,530,101 or 5,585,089, incorporated herein by reference.

In the first structural element, the framework of the heavy chain variable region of the humanized antibody is chosen to have maximal sequence identity (between 65% and 95%) with the framework of the heavy chain variable region of the donor antibody, by suitably selecting the acceptor antibody from among the many known human antibodies. In the second structural element, in constructing the humanized antibody, selected amino acids in the framework of the human acceptor antibody (outside the CDRs) are replaced with corresponding amino acids from the donor antibody, in accordance with specified rules. Specifically, the amino acids to be replaced in the framework are chosen on the basis of proximity to and contact with the CDRs. For example, the replaced amino acids can be adjacent to a CDR in the donor antibody sequence or within 4-6 angstroms of a CDR in the humanized antibody as measured in 3-dimensional space.

A chimeric antibody is a genetically engineered antibody in which the variable region of a mouse (or other rodent) antibody is combined with the constant region of a human antibody. Such antibodies retain the binding specificity of the mouse antibody, while being about two-thirds human. The proportion of nonhuman sequence present in mouse, chimeric and humanized antibodies suggests that the immunogenicity of a chimeric antibody is intermediate between mouse and humanized antibodies. However, some chimeric antibodies have been reported to cause little or no HAMA response in human patients (e.g., LoBuglio et al., Proc. Natl. Acad. Sci. USA 1991:86:4220-4224), such as chRFT5 (Amlot et al., Transplantation 1995:60:748-756).

Other types of genetically engineered antibodies that may have reduced immunogenicity relative to mouse antibodies include but are not limited to single-chain antibodies (Huston et al., Proc. Natl. Acad. Sci. USA 1988:85:5879-5883 and Bird et al., Science 1988:242:423-426); antibody fragments such as Fab, (Fab′)₂ and Fv made using recombinant DNA methods; human antibodies made using phage display methods (Dower et al., WO 91/17271; McCafferty et al., WO 92/001047; and Winter, WO 97/20791) or using transgenic animals (Longerg et al., WO 93/12227; Kucherlapati WO 91/10741); bifunctional antibodies (e.g., PCT/US92/10140); and antibodies with altered constant regions (e.g., U.S. Pat. No. 5,624,821).

A genetically engineered antibody is said to have reduced immunogenicity relative to a mouse antibody from which it is derived, or to be less immunogenic, if when injected into humans or other primate species, it on average causes a reduced HAMA response. That is, the recipient generates less than 2-fold, 5-fold, preferably 10- or 100-fold less titer of antibodies against the injected genetically engineered antibody than against the mouse antibody when similarly administered, as measured by standard assays (see e.g., Hakimi et al., J. Immunol. 1991:147:1352-1359), especially when administered at least 1, 2, 5 or 14 times in a daily, weekly or every other week regimen. The antibody is said to be (essentially) non-immunogenic if when administered at least 1, 2, 5 or 14 times in a daily, weekly or every other week regimen to humans or other primates, few or no (i.e., less than about 10% or 20% but preferably less than 1% or 2%) recipients develop a detectable or significant HAMA response, or a HAMA response that requires cessation of treatment or renders treatment ineffective. For example, humanized anti-Tac has reduced immunogenicity relative to mouse anti-Tac in monkeys (Hakimi et al., supra) and is (essentially) non-immunogenic in human patients. A chimeric antibody to the p55 subunit of the IL-2 receptor antibody, chRFT5, is also non-immunogenic in human patients (Amlot et al., op. cit.).

Pharmaceutical Compositions

For administration to patients, the genetically engineered, chimeric or humanized monoclonal antibody to p55 are typically formulated in a pharmaceutically acceptable carrier. That is, the antibodies can be used in the manufacture of a medicament for treatment of solid organ transplant patients. A variety of aqueous carriers can be used, e.g., water for injection (WFI), or water buffered with phosphate, citrate, acetate, etc. to a pH typically of 5.0 to 8.0, most often 6.0 to 7.0, and/or containing salts such as sodium chloride, potassium chloride, etc. to make isotonic. The carrier can also contain excipients such as human serum albumin, polysorbate 80, sugars or amino acids to protect the active protein. The concentration of fusion protein in these formulations varies widely from about 0.1 to 100 mg/ml but is often in the range 1 to 10 mg/ml. The formulated monoclonal antibody is particularly suitable for parenteral administration, and can be administered as an intravenous infusion or by subcutaneous, intramuscular or intravenous injection.

### EXAMPLE

I. Study Design

This study encompasses 10 patients with disseminated fungal infections who are being treated with an amphotericin B formulation. The trial is a study of Zenapax® (dacluzimab) as a medication for amphotericin B formulation treated patients with serious fungal diseases. After baseline evaluations, patients get treated with Zenapax®. Each patient receives Zenapax® for the same duration as he or she receives an amphotericin B formulation. Each patient gets followed for four weeks after cessation. In further discussions of treatment, ‘amphotericin B’ will refer to “an amphotericin B containing antifungal formulation.”

A. Patient Selection

1. Inclusion Criteria; Patients conforming to the following criteria are considered for the trial:

a. Male or female in-patients at 18-65 years of age.

b. Patients who will receive amphotericin B in the treatment regimen of diagnosed or suspected systemic fungal infections including, but not limited to aspergillosis, histoplasmosis, blastomycosis, coccidiomycosis, cryptococcosis, candidiasis.

c. Life expectancy of a least 1 week.

2. Exclusion Criteria: The following patients will not be enrolled:

a. Patients with other severe acute or chronic medical conditions that in the judgment of the Principal Investigator may limit participation for the full duration of the trial.

b. Patients with a history of poor cooperation, non-compliance with medical treatment or unreliability.

c. Patients with a history of hypersensitivity to monoclonal antibodies or amphotericin B.

d. Patients who have abnormal laboratory values at baseline which are clinically significant for that patient and which may affect the outcome of the study or the interpretation of the results of the study. This includes patients with a calculated CrCl<50 mL/min.

e. Patients who are pregnant, lactating or (+) Pregnancy Test.

f. Patients who have received amphotericin B or Zenapax® within the last 3 weeks.

g. Patient receiving diuretics within 12 hours of enrollment.

B. Concomitant Treatments

There are no specific restrictions regarding concomitant medications for other diagnosis (except as noted in the exclusion criteria) since it is expected that the patients entered into this trial will be receiving other medications. Use of nephrotoxic drugs other than amphotericin B was discouraged since they may contribute to an additive effect and render interpretation of study results difficult. Concomitant medications will be kept to a minimum during the study and recorded at each visit. The investigator will endeavor to maintain the dosages of amphotericin B and essential concomitant medications at a constant level throughout the study.

C. Study Procedures

Patients having a suspected or diagnosed fungal infection and to receive amphotericin B are eligible for entry into the study.

Ten patients with mycotic infections are enrolled to receive Zenapax® 1 mg/kg/q week during their concurrent amphotericin B therapy.

If all inclusion and exclusion criteria are met, the patients will be informed, provide consent and undergo all baseline examinations: history & physical exam, signs and symptoms, blood and urine evaluations and concomitant medication determination before beginning amphotericin B treatment. If available, the values for BUN, creatinine and any other renal function tests performed on the patient within the previous 14 days are noted.

On days 1-7 (if still receiving amphotericin B treatment) the patients have daily urinalysis and evaluation of serum creatinine. On days 7, 14, 21 and 28 the patients (if still receiving amphotericin B treatment) undergo blood and urine determinations, signs and symptoms evaluations, concomitant medication determination and adverse event evaluation.

On the final amphotericin treatment day all evaluations noted above occur and study medication stops. The patient gets evaluated for one more week.

D. Outcome Evaluations

1. Assessment of Signs and Symptoms

a. Interval evaluation of the patient's status and symptoms/signs (vitals), particularly in regard to infection and renal status, made daily after baseline and then four weeks after study.

b. Changes in clinical and microbiological status of the fungal infections.

(1) Comparison regarding the clinical and microbiologic healing or improvement in the fungal infections.

2. Adverse events

a. Amphotericin B induced Nephrotoxicity

(1) Changes in renal function tests: serum blood urea nitrogen and creatinine and calculated glomerular filtration rate (GFR), measured at baseline and then daily; measured GFR performed at baseline, weekly during therapy and 7 days after treatment. Outcome measures for nephrotoxicity get laboratory based.

(2) Adverse reaction profile assessed daily during and 2 hours after drug administration.

b. Amphotericin B Induced Reactions—“Flu-like syndrome”

(1) Patient reactions experienced during amphotericin B infusion get recorded. Specific reactions observed for include: fever, chills, hypotension, headache, respiratory distress and myalgias.

c. The adverse reactions attributable to Zenapax® get monitored to assess increased or altered signs and symptoms associated with combining amphotericin B+ Zenapax®.

(1) Cytokine Release Syndromes

(2) Anti-idiotypic antibody responses

(3) Hypersensitivity reaction

3. Pharmacodynamics of the pharmacologic agents

a. Serum concentrations of amphotericin B measured by either enzyme linked immunoassay or high pressure liquid chromatography for assessment of altered pharmacokinetics secondary to Zenapax® administration.

b. Blood leukocytes (lymphocytes) assessed for changes in IL-2R secondary to amphotericin B administration.

II. Laboratory Determinations

A. Complete Blood Count (CBC) & Serum Chemistry

1. Blood for determination of the CBC and serum chemistry (except SCr) obtained from each patient in a fasting state (if possible) at baseline and weekly for the duration of the study (5 weeks). The CBC include hemoglobin, hematocrit, WBC and differential, RBC count and platelet count along with microscopic evaluation. The serum chemistry include glucose, BUN, albumin, uric acid, calcium, phosphorus, cholesterol, triglycerides, total bilirubin, alkaline phosphatase, LDH, SGOT, SGPT, GGTP, total protein, Na, K, Cl, CO₂, Serum creatitine (Scr) performed daily for the first week in addition to the above studies.

B. Urinalysis and 24 Hour Urine Studies

1. A timed urine collection done at baseline before amphotericin B therapy starts. If the clinical urgency of the situation prevents a 24-hour collection, then a shorter collection. A timed collection of at least two hours attempted. Twenty-four hour collections of urine collected subsequently at days 7, 14, 21, 28 and 35. Determinations include: total volume [glucose, total creatinine (and determination of creatinine clearance), Na, K, Ca, Mg, P and total protein.] The urinalysis includes pH, specific gravity, albumin, glucose, blood, acetone and microscopic evaluation.

2. Measurement of Glomerular Filtration Rate (GFR) at baseline (if possible) and at days 7, 14, 21, 28 and 35.

C. Serum Amphotericin B Levels

1. Blood collected and prepared for determination of serum amphotericin B concentrations on days 1, 7, 14, 21 and 28 prior to drug administration. In addition, on days 7, 14, 21 and 28 a two-hour post study drug dose blood drawn for peak amphotericin levels. The time from last doses of amphotericin along with the time of blood drawing recorded. Amphotericin B concentrations performed by ELISA.

D. IL-2 Receptor Density Studies

1. Blood collected and prepared for determination of serum soluble IL-2 receptor concentrations on days 1, 7, 14, 21 and 28 prior to drug administration. In addition, on days 7, 14, 21 and 28 a two-hour post study drug dose blood collected for determination of sIL-2R concentration. The time from last doses of Zenapax® along with the time of blood drawing recorded. Soluble IL-2R performed by ELISA.

III. Supplies and Drug Accountability

Amphotericin B is supplied as Fungizone® intravenous manufactured by E. R. Squibb and Sons, Inc., Princeton, N.J., or other acceptable pharmaceutical manufacturers.

Interleukin-2 receptor antibody is supplied as Zenapax® intravenous manufactured by Roche Laboratories.

IV. Statistical Analysis

Calculated sample size was based upon a 50% reduction of nephrotoxicity assuming a baseline CrCl of 70 ml/min±7 ml/min in this diseased population and a decrease of greater than 25 ml/min in patients receiving amphotericin B at doses greater than 500 mg.

1. A method for treating a patient having a fungal infection, comprising administering to said patient a therapeutically effective amount of an antifungal agent in association with a therapeutically effective amount of a chimeric or humanized monoclonal antibody that binds to the p55 subunit of the human interleukin-2 (IL-2) receptor and inhibits binding of IL-2 to an IL-2 receptor.

2. The method of claim 1, wherein the infection is a mycotic infection.

3. The method of claim 1, wherein the monoclonal antibody is a humanized anti-Tac antibody.

4. The method of claim 3, wherein the monoclonal antibody is daclizumab.

5. The method of claim 1, wherein the antifungal agent is an antimycotic agent.

6. The method of claim 5, wherein the antimycotic agent is a polyene antimycotic.

7. The method of claim 6, wherein the polyene antimycotic is amphotericin B.

8. The method of claim 1, wherein the antifungal agent is amphotericin B and the monoclonal antibody is daclizumab.

9. The method of claim 8, wherein administration of the therapeutically effective amount of amphotericin B and the therapeutically effective amount of daclizumab occurs concomitantly.

10. The method of claim 8, wherein a part of the therapeutically effective amount of daclizumab is first administered followed by a combination of the remainder of the therapeutically effective amount of daclizumab in association with the therapeutically effective amount of amphotericin B.

11. The method of claim 8, wherein the amphotericin B is administered intravenously.

12. The method of claim 11, wherein the amount of amphotericin B is 0.3 to 25 mg/kg per day.

13. The method of claim 8, wherein daclizumab is administered intravenously.

14. The method of claim 13, wherein the amount of daclizumab is about 1 mg/kg one to four times per week.

15. A method for treating a patient having a fungal infection, comprising administering over a given period of time to the patient (a) a first component consisting of pharmaceutical composition consisting of an injectable solution containing as an active ingredient an antimycotic agent in a therapeutically effective amount to decrease the severity of the fungal infection and (b) a second component consisting of an injectable solution containing as an active ingredient a chimeric or humanized monoclonal antibody that binds to the p55 subunit of the human interleukin-2 (IL-2) receptor and inhibits binding of IL-2 to an IL-2 receptor in a therapeutically effective amount to prevent reactions commonly experienced by patients who receive an antimycotic agent, said components being concomitantly administered over a period of time at least sufficient to resolve an invasive fungal infection.

16. The method of claim 15, wherein the antimycotic agent is amphotericin B.

17. The method of claim 16, wherein second component consists of an injectable solution containing as an active ingredient a humanized anti-Tac monoclonal antibody.

18. The method of claim 17, wherein the anti-Tac monoclonal antibody is daclizumab.

19. A method of treating a patient having a fungal infection, comprising concomitantly administering to the patient:
(i) a first component consisting of an injectable solution containing as an active ingredient amphotericin B, and
(ii) a second component consisting of an injectable solution containing as an active ingredient a chimeric or humanized monoclonal antibody that binds to the p55 subunit of the human interleukin-2 (IL-2) receptor and inhibits binding of IL-2 to an IL-2 receptor.

20. The method of claim 19, wherein the active ingredient of the second component is a humanized anti-Tac antibody.

21. The method of claim 19, wherein the active ingredient of the pharmaceutical composition of the first component is administered in an approximate amount of 0.3 to 25 mg per kg per day.

22. The method of claim 19 wherein the active ingredient of the second component is administered in an approximate amount of 1 mg/kg to 2 mg/kg per week.

23. The method of claim 19, wherein the components are concomitantly administered over a period of time from about 4 weeks to about 8 weeks.

24. The method of claim 23, wherein the components are concomitantly administered over a period of time of about 8 weeks.

25. A kit comprising:
(a) a first component containing a vial or series of vials, each vial containing a single injectable solution dose or multiple injectable solution doses, each dose containing an active ingredient about 50 mg to about 100 mg of the active ingredient, wherein the active ingredient is amphotericin B, and
(b) a second component containing a vial or series of vials, each vial containing a single injectable solution dose or multiple injectable solution doses, each dose containing as an active ingredient about 1 mg/kg to about 2 mg/kg of a chimeric or humanized monoclonal antibody that binds to the p55 subunit of the human interleukin-2 (IL-2) receptor and inhibits binding of IL-2 to an IL-2 receptor.

26. The kit of claim 25, wherein the first component contains a sufficient number of units so that a patient can administer about 0.3-25 mg per kg of amphotericin B daily for a period of about 4 to about 8 weeks and the second component contains a sufficient number of doses so that a patient can administer about 1-2 mg per kg of the monoclonal antibody weekly for a period of about 4 to about 8 weeks.

27. The kit of claim 25, wherein the monoclonal antibody of each injectable solution dose of the second component is a humanized anti-Tac antibody.

28. The kit of claim 27, wherein the humanized anti-Tac antibody is daclizumab.

29. The kit of claim 25, wherein the first component contains a sufficient number of units so that a patient can administer about 0.3-25 mg per kg of amphotericin B daily for a period of about 4 to about 8 weeks and the second component contains a sufficient number of doses so that a patient can administer about 1 mg/kg weekly of the monoclonal antibody for a period of about 4 to about 8 weeks.

30. A method for treating a patient having a fungal infection, comprising administering to the patient over a period of time at least sufficient to resolve an invasive fungal infection, in fourteen day cycles, (a) a first component, delivered on day 1 of each fourteen day cycle, consisting of an injectable solution containing as an active ingredient a chimeric or humanized monoclonal antibody that binds to the p55 subunit of the human interleukin-2 (IL-2) receptor and inhibits binding of IL-2 to an IL-2 receptor in a therapeutically effective amount to prevent reactions commonly experienced by patients who receive an antifungal agent, and (b) a second component, delivered each day of each fourteen day cycle, consisting of pharmaceutical composition consisting of an injectable solution containing as an active ingredient an antifungal agent in a therapeutically effective amount to decrease the severity of the fungal infection.

31. The method of claim 30 wherein said antifungal agent is amphotericin B.

32. The method of claim 30 wherein said monoclonal antibody is an anti-Tac antibody.

33. The method of claim 32 wherein said anti-Tac antibody is daclizumab.

34. The method of claim 30 wherein the monoclonal antibody is administered in an amount of about 1 mg/kg to about 2 mg/kg weekly.

35. The method of claim 30 wherein the antimycotic agent is administered in an amount of about 0.3-25 mg/kg daily.

36. The method of claim 30 wherein components (a) and (b) are administered for a period of about 4 to 8 weeks.

37. A method of reducing side effects associated with antifungal therapy selected from the group consisting of renal dysfunction with secondary hypokalemia, hypomagnesemia and anemia, flu-like syndrome, epigastric pain, cardiovascular toxicity, hypotension, ventricular fibrillation, cardiac arrest, pulmonary leukocytosis, dyspnea, respiratory distress, hepatic dysfunction or failure, coagulation defects, pruritis, seizures, and nephrotoxicity comprising administering to a patient a therapeutically effective amount of a chimeric or humanized monoclonal antibody that binds to the p55 subunit of the human interleukin-2 (IL-2) receptor and inhibits binding of IL-2 to an IL-2 receptor.

38. The method of claim 37 wherein the monoclonal antibody is an anti-Tac antibody.

39. The method of claim 38 wherein the anti-Tac antibody is daclizumab.

40. The method of claim 37 wherein the monoclonal antibody is administered in an amount of 1 mg/kg to 2 mg/kg/week.

41. The method of claim 37 wherein the monoclonal antibody is administered in an injectable solution.
